(19) United States
(12) Patent Application Publication
(10) Pub. No.: US 2012/0267322 A1
(43) Pub. Date: Oct. 25, 2012

(54) METHOD AND SYSTEM FOR PLASMA TREATMENT OF A LIQUID

(75) Inventors: Reuven Lev Boxman, Herzliya (IL); Vladimir Dikhtiar, Tel Aviv (IL); Olga Dikhtyar, Ashdod (IL); Nahum Parkansky, Raanana (IL)

(73) Assignee: Ramot At Tel-Aviv University Ltd., Tel Aviv (IL)

(21) Appl. No.: 13/498,360

(22) PCT Filed: Oct. 3, 2010
(86) PCT No.: PCT/IL10/00800

§ 371 Date: Jun. 18, 2012

Related U.S. Application Data
(60) Provisional application No. 61/272,573, filed on Oct. 7, 2009.

(51) Int. Cl. A61L 2/14; H05B 6/74; C02F 1/32; C02F 9/12; B82Y 40/00

(52) U.S. Cl. 210/748.11; 250/435; 210/748.16; 219/745; 977/903

(57) ABSTRACT

A system for plasma treatment of a liquid and corresponding uses thereof, the system comprising a chamber having a liquid input conduit and a liquid output conduit, a source of electromagnetic radiation of wavelength λ coupled to the chamber by a waveguide of appropriate width to provide only fundamental mode of the electromagnetic radiation, a focusing means positioned between the waveguide and the chamber for transmissively focusing the electromagnetic radiation incident thereon to a location within the chamber remote from walls of chamber for creating a plasma at the location, characterized in that the plasma is formed entirely within the liquid.

### FIELD OF THE INVENTION

The present invention is directed to a system and associated method for creating plasma within a liquid, particularly for treating water by decomposing organic molecules in general and destroying pathogens in particular, but also for creating nanoparticles in simple organic liquids such as ethanol.

### BACKGROUND

It is known that plasma treatment of water can have a sterilizing effect that is attributed to the microbiocidal effects of intense radiation, particularly of ultraviolet light, and to the oxidation effects of various chemical species, particularly peroxide and ozone. Similarly, it is well established that nanoparticles of carbon, such as C₆₀ Buckminster fullerene and more complex nested fullerenes, may be synthesized in simple organic liquids like ethanol by creating bubbles of plasma therein.

Dielectric breakdown followed by an arc discharge is one method that has been used to create plasma within a liquid but ablation of the electrodes by the plasma is a non-desirable side effect that contaminates the liquid and causes a need for frequent electrode replacement. The problems of electrode ablation are discussed in “Discharge Characteristics of Microwave and High-Frequency In-Liquid Plasma in Water” Applied Physics Express 1 (2008) 046002 by Nomura et al. Their solution is to use a titanium electrode covered with ceramic, with only the tip thereof exposed. However, it will be appreciated that at least this exposed tip may be subject to considerable electrode erosion (ablation), shortening the Mean Time Between Failures (MTBF), contaminates the treated water, and may preclude using such a device for many purposes. Also, practical difficulties were experienced with the feedthrough of the electrode and the surrounding insulator with insulator cracking and/or melting occurring.

Another approach has been to use a slit opening in a waveguide as a plasma source. See T. Ishijima et al., “Multibubble plasma production and solvent decomposition in water by slot-excited microwave discharge” Applied Physics Letters 91, 121501 2007″. Here the sides of the slit re ablated.

It would be desirable to create bubbles of plasma within a liquid without exposing additional materials, such as electrodes to the plasma, particularly for treating water. It would be desirable to produce a submerged plasma discharge without any electrode, and hence without introducing any contaminants into the treated liquid. The present invention addresses this need.

### SUMMARY OF THE INVENTION

The present invention is directed to providing a system for plasma treatment of a liquid comprising a chamber having a liquid input conduit and a liquid output conduit; a source of electromagnetic radiation of wavelength λ coupled to the chamber by a waveguide of a width to provide only the fundamental mode of the electromagnetic radiation; a focusing means positioned between the waveguide and the chamber for transmissively focusing the electromagnetic radiation of wavelength λ incident thereon to a location within the chamber remote from walls of chamber for creating a plasma at the location, characterized that the plasma is formed entirely within the liquid.

In one embodiment, the liquid is water and the focusing means is a section of a material having a dielectric constant of 8.9 between the chamber and the waveguide, and serving as a converging lens.

Preferably, the surface of the chamber opposite to the waveguide is reflective to reflect radiation back into the chamber.

Preferably, the surface of the chamber opposite to the waveguide is concave and reflects and focuses radiation back towards the location.

Preferably, the chamber has a curved surface adjacent to the waveguide.

Preferably, the chamber has a curved surface opposite the waveguide.

Preferably, the chamber is substantively spherical and the location is substantively central to the chamber.

In a preferred embodiment, the system further comprises a gas inlet in the base of the chamber coupled by a conduit to a gas source for controlled bubbling of a gas into the chamber and an outlet for allowing the gas to exit the chamber.

In a preferred embodiment, the water inlet conduit is in lower section of the chamber and the output conduit is opposite to the gas inlet so that bubbles are vented from chamber thereby.

Preferably, the inner surface of the chamber is characterized by at least one of:

(i) a reflective surface for reflecting electromagnetic radiation, and

(ii) an oxidation resistance surface.

In a preferred embodiment, at least part of the inner surface is coated with a photocatalytic material.

Optionally, the photocatalytic material is selected from the group consisting of TiO₂ and N-doped TiO₂.

In one application, the system is used for treatment of water, said treatment being a sanitization treatment comprising decomposition of harmful organic pollutants.

In a second application, the system is used for treatment of organic liquid, where said treatment is formation of carbon nanoparticles.

In one embodiment, the electromagnetic radiation is microwave radiation and the source of electromagnetic radiation is a magnetron coupled to an antenna protruding into the waveguide.

In a second aspect, the present invention is directed to providing a method of sanitizing water comprising the steps of:
(a) Providing a chamber having a liquid input conduit and a liquid output conduit; a source of electromagnetic radiation of wavelength λ coupled to the chamber by a waveguide of corresponding appropriate width to provide only fundamental mode of the electromagnetic radiation; a focusing means positioned between the waveguide and the chamber for transmissively focusing the electromagnetic radiation of wavelength λ incident thereon to a location within the chamber remote from walls of chamber for creating a plasma at the location,
(b) supplying electromagnetic radiation from the source and
(c) creating a plasma bubble within the water, thereby exposing any microbes and/or pathogens within the water to sterilizing effects of at least one of the group comprising UV light, ozone, hydrogen radicals and hydroxyl radicals.

In a third aspect, the present invention is directed to a method of forming carbon nanoparticles by the steps of:

(i) providing a chamber having a liquid input conduit and a liquid output conduit; a source of electromagnetic radiation of wavelength λ coupled to the chamber by a waveguide of a width to provide only fundamental mode of the electromagnetic radiation; a focusing means positioned between the waveguide and the chamber for transmissively focusing the electromagnetic radiation of wavelength λ incident thereon to a location within the chamber remote from walls of chamber for creating a plasma at the location,

(ii) introducing an organic liquid into the chamber and

(iii) creating a plasma bubble within the organic liquid.
By fundamental mode, the mode with the lowest frequency which propagates in a waveguide is intended. With proper choice of waveguide dimensions and frequency, only the fundamental mode propagates, while higher-order modes decay along the length of the waveguide.

### DESCRIPTION OF THE FIGURES

For a better understanding of the invention and to show how it may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention; the description taken with the drawings making apparent to those skilled in the art how the several forms of the invention may be embodied in practice. In the accompanying drawings:

FIG. 1 is a schematic cross-sectional view of one embodiment of a system for plasma treatment of a liquid;

FIG. 2 shows the geometry used in a computer simulation of generation of plasma within the system substantially as illustrated in FIG. 1;

FIG. 3, using the computer simulation, shows that the maximum field occurs at approximately 72°;

FIG. 4, using the computer simulation, shows a vector representation of the E-field at 85°;

FIG. 5, using the computer simulation, shows a simulation of the specific Absorption Rate;

FIG. 6, using the computer simulation, shows the average specific absorption rate;

FIG. 7, using the computer simulation, shows the E-field on y-z plane, f=2.45 GHz, 168°, with a 10 mm bubble;

FIG. 8, using the computer simulation, shows the E-field magnitude for f=2.45 GHz, phase=132, R_{bubble}=1 mm;

FIG. 9, using the computer simulation, shows the E-field vector plot for f=2.45 GHz, 132°, R_{bubble}=1 mm;

FIG. 10, using the computer simulation, shows the E-field vector plot for f=2.45 GHz, 132°, R_{bubble}=1 mm for an expanded view of the bubble region;

FIG. 11, using the computer simulation, shows the Average SAR, f=2.45 GHz, R_{bubble}=1 mm, and

FIG. 12 shows the steps of a method of purifying water using the system of FIG. 1.

### DESCRIPTION OF PREFERRED EMBODIMENTS

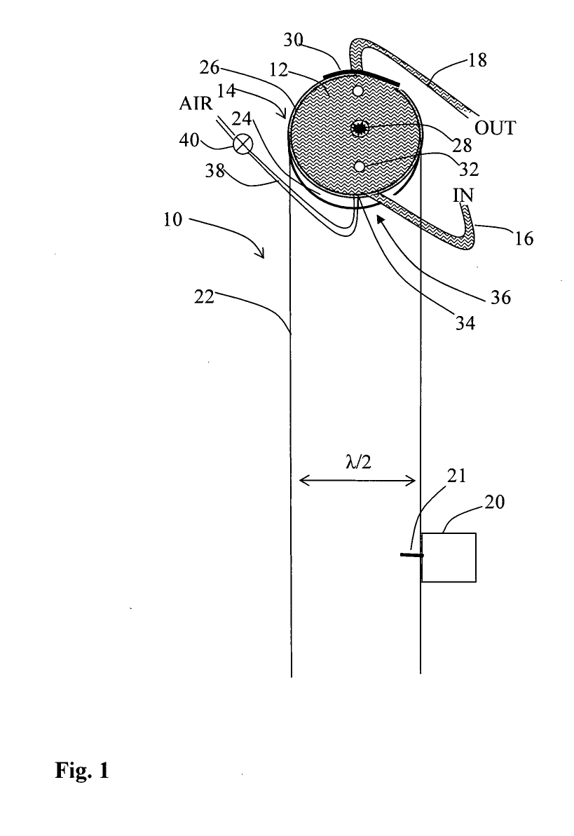

With reference to FIG. 1, a system 10 for plasma treatment of a liquid 12 is shown. The system 10 comprises a chamber 14 having a liquid input conduit 16 and a liquid output conduit 18. A source of electromagnetic radiation 20 of wavelength λ coupled to the chamber 14 by a waveguide 22 of diameter D. A focusing means 24 is positioned between the waveguide 22 and the chamber 14 for transmissively focusing the electromagnetic radiation of wavelength λ incident thereon, to a location within the chamber 14, that is remote from walls 26 of the chamber 12 for creating a bubble of plasma 28 at the location, such that the bubble of plasma 28 is formed entirely within the liquid 12. The focusing means 24 may be a section of high dielectric material situated between the chamber 14 and the waveguide 22, and serves as a converging lens for transmissively focusing radiation of wavelength λ onto a discrete location, typically near the center of the chamber 14.

Preferably the surface 30 of the chamber 14 opposite to the waveguide is reflective to reflect radiation back into the chamber 14, and more preferably, the surface 30 of the chamber 14 opposite to the waveguide 22 is concave and reflects and focuses radiation back towards the location.

In the embodiment shown, the chamber 14 is substantively spherical and the location is substantively central to the chamber. It will be appreciated that other curved surfaces such as cylindrical and parabolic surfaces adjacent to the waveguide and opposite thereof will work to a greater or lesser extent, so sphericity is not critical.

If sufficient energy is focused at a small location X, the liquid 12 may be broken down to form a bubble of plasma. To conserve energy it is preferable to introduce a bubble 32 of gas into the liquid and thus optionally and preferably, the 10 further comprises a gas inlet 34 in the base 36 of the chamber 14 that is coupled by a conduit 38, to a gas source via a valve 40 for controlled bubbling of a gas into the chamber 14. In this manner, bubbles 32 may be introduced into the chamber 14 and plasma 28 may be formed with a bubble 32 at location X, requiring less energy than that needed to create a bubble of plasma 28 within the liquid. Other methods of creating bubbles are possible of course, including boiling the liquid. Indeed, in this regard as described below, in one experimental proof of concept, a magnetron 20 from a microwave oven having an antenna 21 protruding into the waveguide 22 may be used to create microwaves. It will be appreciated that microwave energy excites water molecules and can cause boiling and this system may generate bubbles of water vapor for converting into plasma 28.

It will be appreciated that the operating conditions, such as, inter alia, power, water and gas flow rates, may be adjusted so that that plasma within the bubbles will be deionized and converted into neutral gaseous species before the bubbles 32 hit the walls 26 of the chamber 14. In preferred embodiments, the placement and size of the outlet 18 may be chosen so that any plasma bubbles produced will be contained within the liquid flowing through the outlet 18. In either of these embodiments, no material other than the liquid 12 within the chamber 14 comes into contact with the plasma 28 and so plasma ablation is totally avoided. Since bubbles 32 rise, it is preferably that bubble inlet conduit 38 be situated in the lowest point of the chamber 14 and that the liquid outlet 18 be situated directly opposite, at the topmost side of the chamber 14, and should continue vertically for a few centimeters, thereby providing additional time for the plasma conditions within the bubble 32 to cease, and the bubble to return to being a simple gas bubble or to implode.

There are a number of mechanisms by which the system 10 may deactivate pathogens and decompose organic pollutants. For example, plasma emits intense electromagnetic radiation, including UV radiation that is damaging to microbiological life-forms. Ozone, free radicals and peroxide species may be formed that are powerful oxidizers which attack the protective skins of microbes and viruses. It is preferable that the wall 26 of the chamber 14 has a reflective surface thereupon for reflecting such electromagnetic radiation back into the chamber. Such a coating should, itself, be oxidation resistant. It is preferable that wall 26 of chamber 14 have a photocatalytic surface. In this manner radiation emitted by the plasma 28 and not absorbed in the liquid 12 may be utilized by the photocatalytic surface to directly decompose pollutant molecules contacting it or to create OH radicals from water molecules; it being appreciated that OH radicals may serve as a powerful oxidizer which may damage microbial membranes and decompose pollutant molecules. One appropriate photocatalytic material may be a thin film of titanium—oxide TiO₂, whose photocatalytic activity is activated by UV radiation, or N-doped TiO₂, which can be activated by less energetic photons, depending on the dopant concentration even by visible radiation,

### Specific Embodiment

With reference to FIG. 1, in one system the electromagnetic radiation source 20 is a microwave generator consisting of a 2.45 GHz magnetron from a domestic microwave oven and having a monopole antenna 21 mounted directly on the magnetron 20, which protrudes into the waveguide 22. Compatible with the microwaves emitted by the magnetron 20, a 80 mm diameter circular waveguide, with a conducting wall is used. The focusing means 24 provided is a dielectric matching element 24 having a refractive index of k=8.9 and two parallel spherical surfaces, with radii of curvature 40 mm and thickness 10 mm, and the chamber is a spherical water chamber, R=40 mm, bounded on one side by the concave surface of the dielectric matching element, and on the other by a conductor with a concave spherical profile. The inside surface of the chamber (excluding the dielectric matching element and conductor on the opposite face) is coated with TiO₂.

The device operates as follows: the magnetron 20 and its antenna 21 radiate microwaves into the circular waveguide 22, which is sized to support only its fundamental TE₁₁ mode. The radiation impinges on the dielectric matching element 24, which, due to the thickness and dielectric constant k thereof, serves as an antireflective coating for the spherical water surface, i.e. its wave impedance is Z_{d}=(η₀Z_{w})^{1/2}, where η₀ is the wave impedance of free space (377 Ω), and Z_{w}=η₀/k^{1/2}=42 Ω is the wave impedance of the water, and where the thickness is −λ/4 in the dielectric. One appropriate material for the dielectric matching element 24 is “Rogers TMM 10 material” The Rogers TMM 10 material laminate may have to be constructed or machined to order to provide a spherical curvature.

The spherical surface of the water refracts the radiation and focuses it to a point location X approximately L=R/(n_{w}−1)=5 mm behind the center of the sphere (where R is the radius of curvature of water surface, and n_{w} is the “RF index of refraction” of the water: n_{w}=k_{w}^{1/2}=9, where k_{w}=81 is the dielectric constant of water. The propagating wave diverges beyond that point, and is focused by the metal reflector 30 back towards the central region of the chamber 14.

It will be appreciated that since the dimensions of the apparatus 10 are comparable to a wavelength of the radiation λ used therein, the performance of the apparatus 10 must be calculated by solving Maxwell's equations, subject to appropriate boundary conditions. It is not practical to do so analytically, but the system was modeled numerically using the Ansoft HFSS™ program, as described below.

Simulation

1. No bubble, f=2.54 GHz (sic)

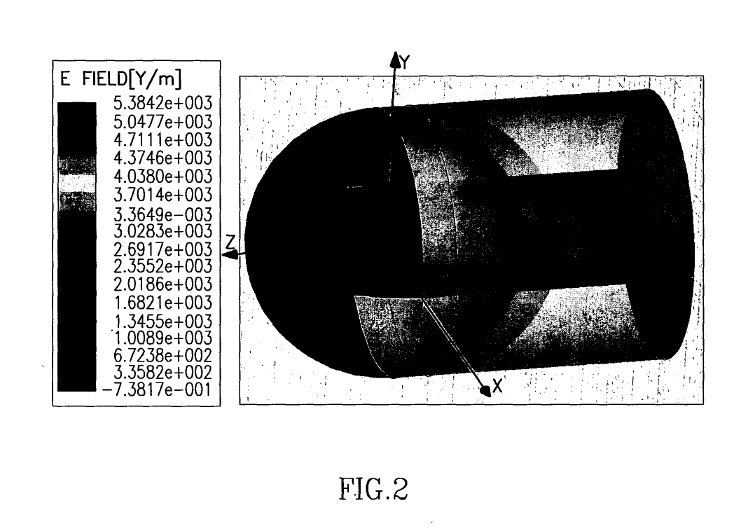

The proposed geometry was simulated using HFSS v. 9 at f=2.54 GHz (sic), but without the magnetron 20 and its connection. For simplicity, the excitation was via a “waveport” at the end of the waveguide 22. In HFSS, the “waveport” is excited by 1 W incident power. The geometry is illustrated in FIG. 2. below. The dielectric matching element 24 is constructed in the simulation with a material designated “Rogers TMM 10”, which is listed in the HFSS library as having k=9.2 and 0 bulk conductivity. Other materials with similar dielectric constants and bulk conductivity may be used.

With respect to FIG. 2, from right to left, waveguide, dielectric matching section, and spherical water chamber are shown. Electric field strength at 0° phase is shown on the x-z plane. The “hot spots” along the axis in the water chamber will be noted.

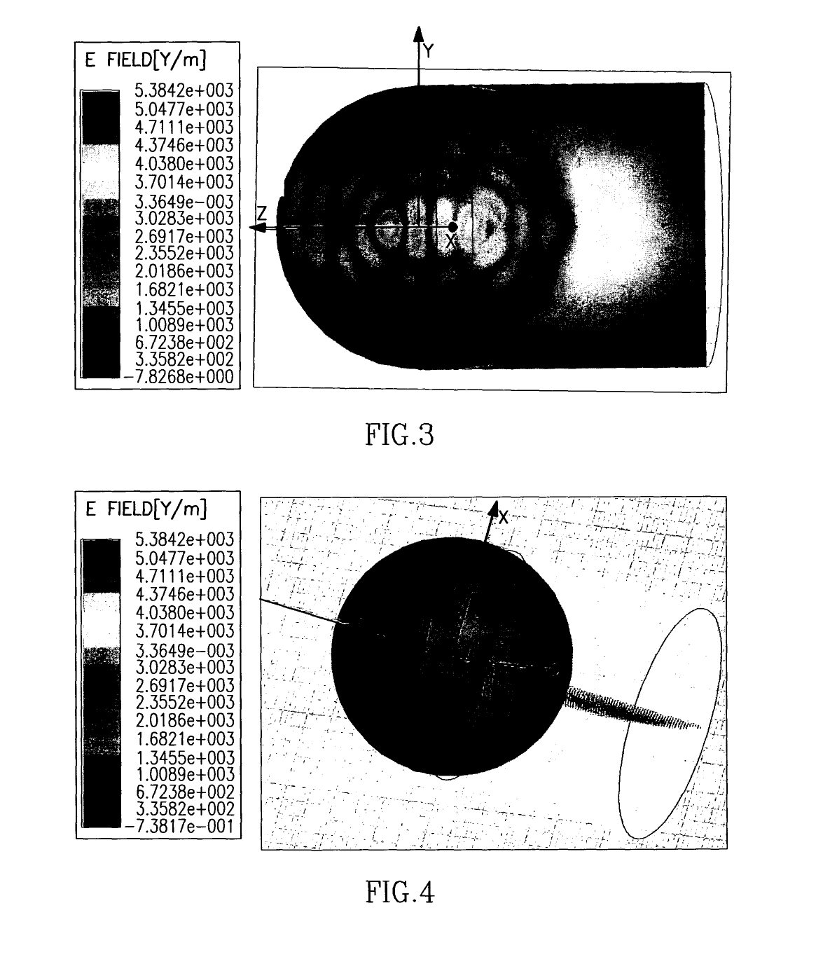

With reference to FIG. 3, the maximum field occurs at approximately 72°, and is illustrated in the x-z plane, regarding which it will be noted that all field magnitude must be scaled according to the actual excitation.

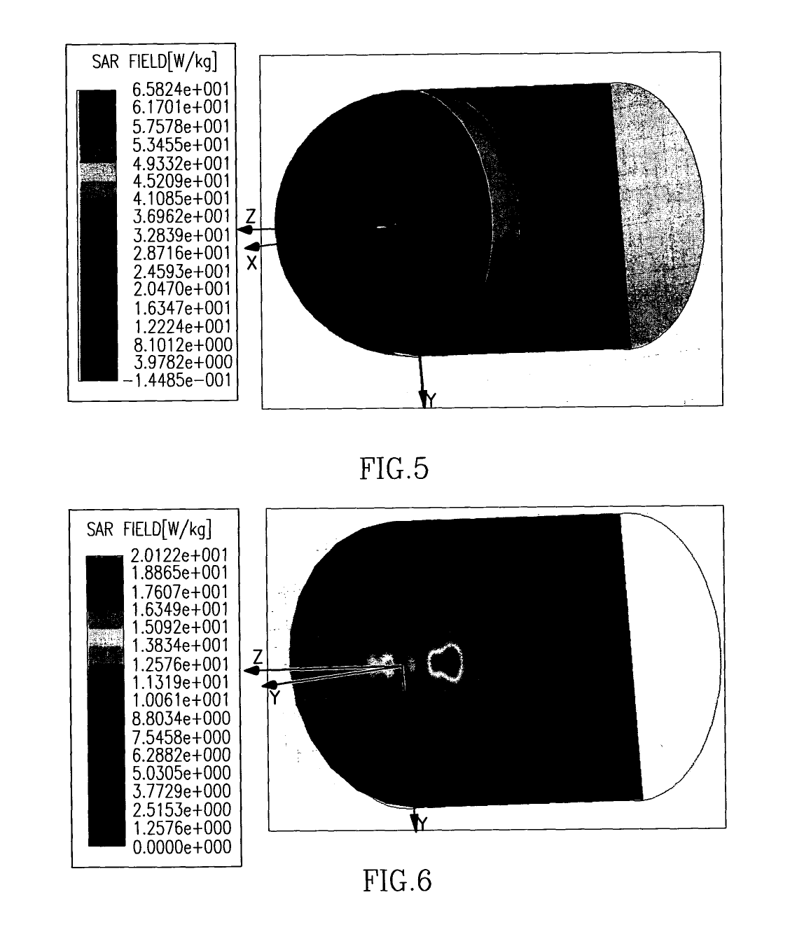

A vector representation of the E-field is shown in FIG. 4 and the absorbance in the water is illustrated in FIG. 5.

Note that the SAR should scale linearly with the input power. At P=1 kW, the SAR in the hot spots should be about 6×10⁴ W/kg. Thus if the water were stationary, the time to heat the water in the hot spot to boiling is Δt=ΔT c/SAR ˜6 s. After reaching the boiling point, a vapor bubble should appear near the hot spot, which will change the field distribution.

FIG. 6 shows the average specific absorption rate. Again, hot spots lying along the axis of the system, near the center of the sphere, are visible.

2. f=2.45 GHz, 10 mm radius bubble at center

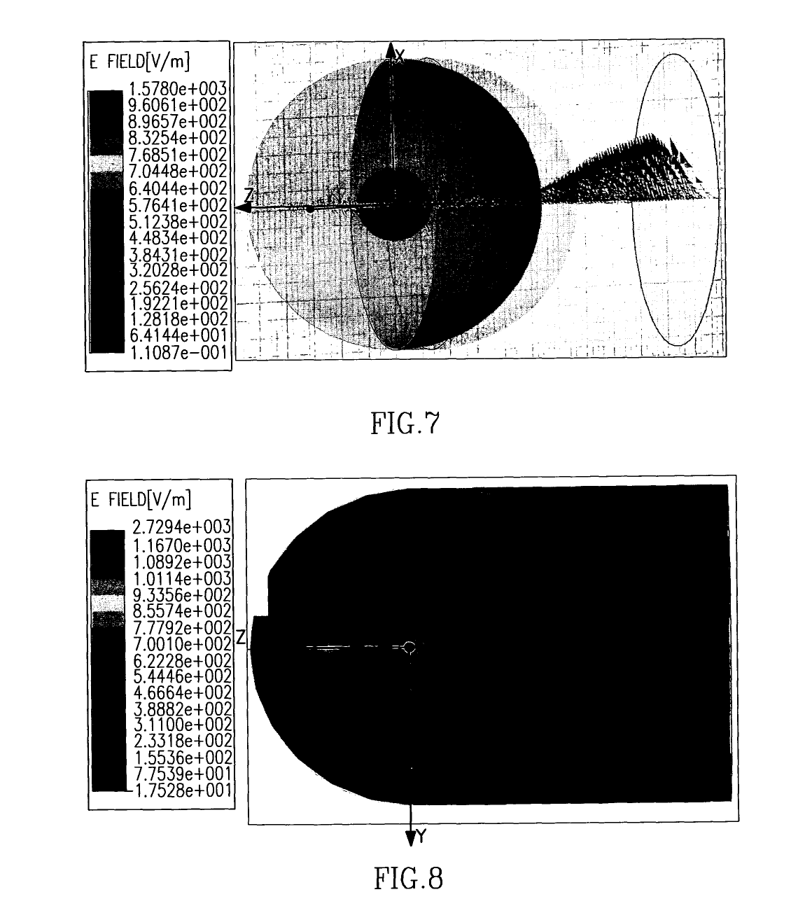

A 10 mm air bubble was inserted at the center of the geometry simulated in the previous section. FIG. 7 shows the vector E-field on the y-z plane, at the time of maximum)field.(168°). It may be seen that the field in the water sphere is considerably weaker than in the previous simulations (no bubble, 2.54 GHz), and that the field is strongest adjacent to the waveport.

3. f=2.45 GHz, 1 mm radius bubble at center

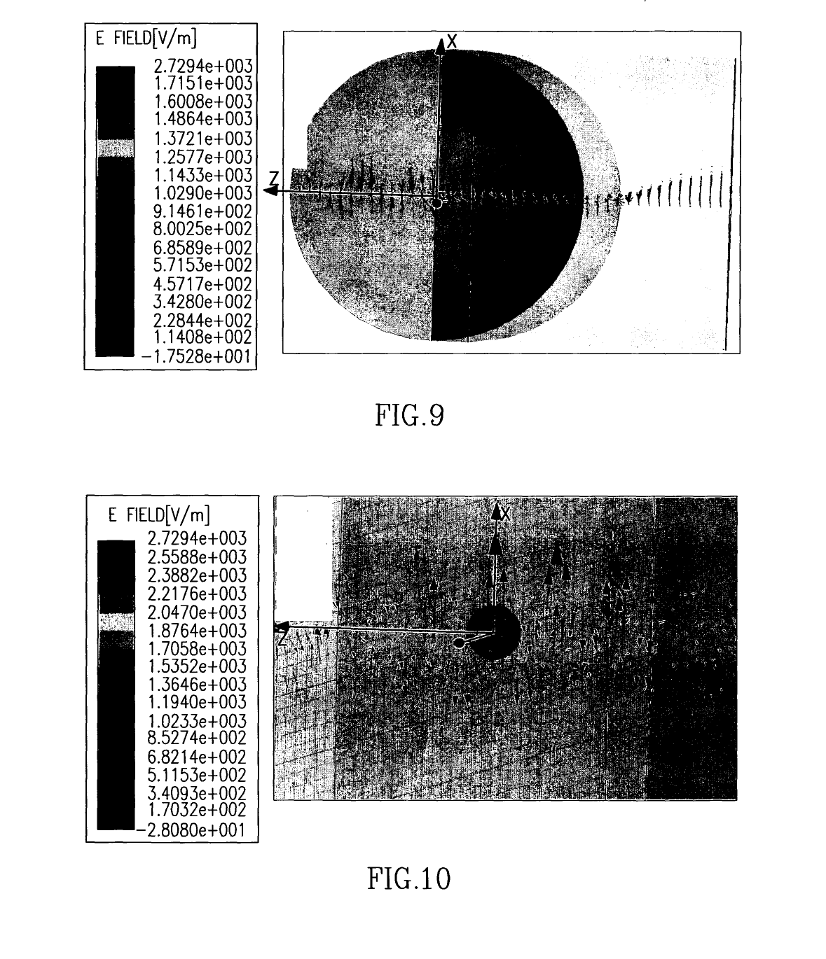

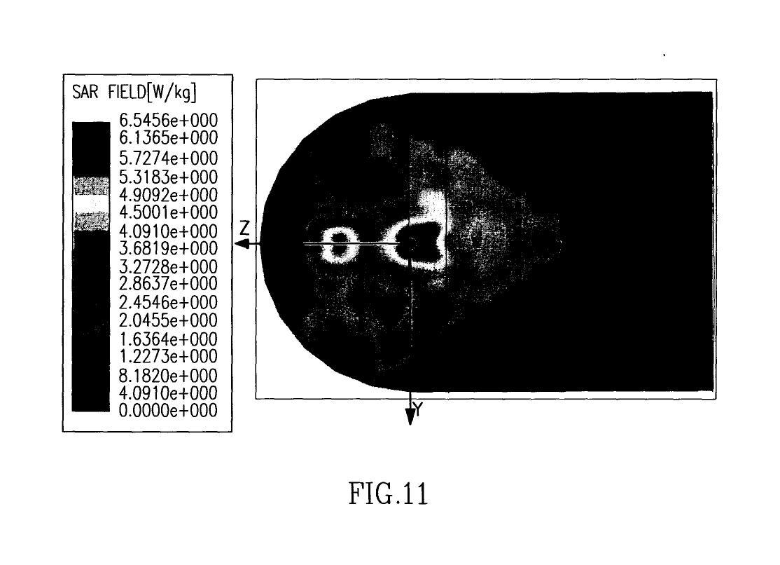

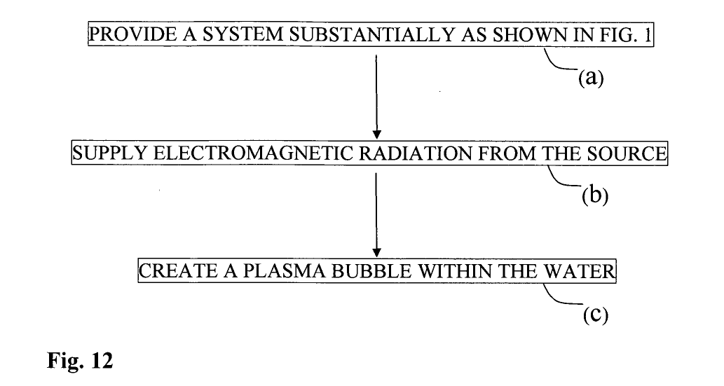

A further simulation was run at f=2.45 GHz, but with a 1 mm bubble. The E-field magnitude and E-field vectors are plotted in FIGS. 8 and 9 below. It may be seen that the maximum E-field appears in the middle of the sphere, i.e. in the bubble. A magnified view of the bubble region is shown in FIG. 10 below. It may be seen that the maximum field is in the bubble, but it is not clear if the grid spacing in the simulation was sufficient to accurately account for the details at the bubble boundary. The average SAR is shown in FIG. 10 below.

Attempts were made to run simulations of the above geometry with higher resolution in the vicinity of the bubble, i.e. for 0.1, 0.15, and 3 mm spacing. All these attempts failed, and HFSS returned an error message that there was insufficient memory.

Thus a system and method for the sanitization treatment of water has been described. The method uses the apparatus 10 of FIG. 1 to create a plasma bubble 28 within the water 12, thereby exposing any microbes and/or pathogens within the water 12 to sterilizing effects such as exposure to UV light, ozone, hydrogen radicals and hydroxyl radicals. It will be appreciated that the various tissue destroying effects work in parallel and possibly synergistically. thus it will be appreciated that the operating conditions, such as water flow rate, power, and the like, may be adjusted to provide an appropriate sterilizing effect.

One embodiment uses a microwave magnetron 20 of the type that is a standard component of domestic microwave ovens, and may be coupled to and is suitable for treating a domestic mains water supply for a single family dwelling. The initial cost of such a system is only tens of dollars and the operating costs are low. The system can, however, be scaled up, using other frequencies and appropriate waveguides for treating water mains for a town, for example, or for sewage treatment. For example, a system could be constructed to operate using the principles described about at a frequency of 245 MHz and a waveguide and water chamber diameter of 0.8 m. Such a system, with a larger water chamber and operating with higher power, could accommodate a higher water flow, and furthermore less of the power would be dissipated heating water since the absorption of water at lower frequencies is less than at 2.45 GHz.

Additionally, it has been found that the system can be used to create carbon nanoparticles from a hydrocarbon containing liquid such as ethanol and the like.

One embodiment has been described in some detail. The scope of the present invention goes beyond this specific embodiment, and the invention is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

1. A system for plasma treatment of a liquid comprising a chamber having a liquid input conduit and a liquid output conduit;
a source of electromagnetic radiation of wavelength λ coupled to the chamber by a waveguide of width to provide only fundamental mode of the electromagnetic radiation,
a focusing means positioned between the waveguide and the chamber for transmissively focusing the electromagnetic radiation of wavelength λ incident thereon to a location within the chamber remote from walls of chamber for creating a plasma at the location, characterized that the plasma is formed entirely within the liquid.

2. The system of claim 1 wherein the liquid is water and the focusing means is a material having 8<k<10 and 0 bulk conductivity, between the chamber and the waveguide, and serving as a converging lens.

3. The system of claim 1 wherein the liquid is water and the focusing means is a material having 9<k<9.5 and 0 bulk conductivity, between the chamber and the waveguide, and serving as a converging lens.

4. The system of claim 1 wherein the surface of the chamber opposite to the waveguide is reflective to reflect radiation back into the chamber.

5. The system of claim 1, wherein the surface of the chamber opposite to the waveguide is concave and reflects and focuses radiation back towards the location.

6. The system of claim 1 wherein the chamber has a curved surface adjacent to the waveguide.

7. The system of claim 1 wherein the chamber has a curved surface opposite the waveguide.

8. The system of claim 1 wherein the chamber is substantively spherical and the location is substantively central to the chamber.

9. The system of claim 1 further comprising a gas inlet in the base of the chamber coupled by a conduit to a gas source for controlled bubbling of a gas into the chamber and an outlet for allowing the gas to exit the chamber.

10. The system of claim 9 wherein the water inlet conduit is in lower section of the chamber and the output conduit is opposite to the gas inlet so that bubbles are vented from chamber thereby.

11. The system of claim 1, wherein the inner surface of the chamber is characterized by at least one of:
(i) a reflective surface for reflecting electromagnetic radiation,
(ii) an oxidation resistance surface, and
(iii) a photocatalytic surface.

12. The system of claim 1 wherein the inner surface of the chamber is coated with a material selected from the group consisting of TiO₂ and N-doped TiO₂.

13. The system of claim 1 for treatment of water, where said treatment is a disinfection treatment comprising killing pathogens.

14. The system of claim 1 for treatment of water, where said treatment is a decomposition of harmful organic pollutants.

15. A system of claim 1 for treatment of organic liquid, where said treatment is formation of carbon nanoparticles.

16. The system of claim 1 wherein the electromagnetic radiation is microwave radiation and the source of electromagnetic radiation is a magnetron coupled to an antenna protruding into the waveguide.

17. A method of disinfecting water within the chamber of claim 1 by creating a plasma bubble within the water, thereby exposing any microbes and/or pathogens within the water to sterilizing effects of at least one of the group comprising UV light, ozone, hydrogen radicals and hydroxyl radicals.

18. A method of treating water within the chamber of claim 1 by creating a plasma bubble within the water, thereby exposing any pollutant molecules within the water to at one of effects comprising oxidation and decomposition of at least one of the group consisting of UV light, ozone, hydrogen radicals and hydroxyl radicals.

19. A method of forming carbon nanoparticles within the chamber of claim 1 by introducing an organic liquid into the chamber and creating a plasma bubble within the organic liquid.
